SEQUENCE LISTING (68 sequences)

SEQ ID NO: 1 - RNA, 21 nt, Homo sapiens
aauuacagaggaaaacuggca

SEQ ID NO: 2 - RNA, 21 nt, Homo sapiens
aggaauuacagaggaaaacug

SEQ ID NO: 3 - RNA, 21 nt, Homo sapiens
auaacugagauguaaugacug

SEQ ID NO: 4 - RNA, 21 nt, Homo sapiens
auguguuuucuucuauaacug

SEQ ID NO: 5 - RNA, 21 nt, Homo sapiens
auaacucugagaaacaugguu

SEQ ID NO: 6 - RNA, 21 nt, Homo sapiens
aauaacucugagaaacauggu

SEQ ID NO: 7 - RNA, 21 nt, Homo sapiens
uguauguuuuuauaugugggg

SEQ ID NO: 8 - RNA, 21 nt, Homo sapiens
ucuuguauccuuuuguugcug

SEQ ID NO: 9 - RNA, 21 nt, Homo sapiens
uucuuguauccuuuuguugcu

SEQ ID NO: 10 - RNA, 21 nt, Homo sapiens
uaucaacacuguuauugucau

SEQ ID NO: 11 - RNA, 21 nt, Homo sapiens
uuaaacuugauaucaacacug

SEQ ID NO: 12 - RNA, 21 nt, Homo sapiens
aaaaggaacguuaaacuugau

SEQ ID NO: 13 - RNA, 21 nt, Homo sapiens
uucaaaaggaacguuaaacuu

SEQ ID NO: 14 - RNA, 21 nt, Homo sapiens
uuaucuugacaccaauuucaa

SEQ ID NO: 15 - RNA, 21 nt, Homo sapiens
ucuugauacucucuuucuguu

SEQ ID NO: 16 - RNA, 21 nt, Homo sapiens
uuuuuguccgaguuucuucag

SEQ ID NO: 17 - RNA, 21 nt, Homo sapiens
agcuuuuuguccgaguuucuu

SEQ ID NO: 18 - RNA, 21 nt, Homo sapiens
uauaucaggccaucuagugcu

SEQ ID NO: 19 - RNA, 21 nt, Homo sapiens
aaguaaaacuuauaucaggcc

SEQ ID NO: 20 - RNA, 21 nt, Homo sapiens
aaaguaaaacuuauaucaggc

SEQ ID NO: 21 - RNA, 21 nt, Homo sapiens
uuaaaguaaaacuuauaucag

SEQ ID NO: 22 - RNA, 21 nt, Homo sapiens
uuuuguuuuaaaguaaaacuu

SEQ ID NO: 23 - RNA, 21 nt, Homo sapiens
ucauaguuuuguuuuaaagua

SEQ ID NO: 24 - RNA, 21 nt, Homo sapiens
aaauucagucauaguuuuguu

SEQ ID NO: 25 - RNA, 21 nt, Homo sapiens
uucuuagagaucuaaauucag

SEQ ID NO: 26 - RNA, 21 nt, Homo sapiens
uucuuucuucuuagagaucua

SEQ ID NO: 27 - RNA, 21 nt, Homo sapiens
aaguucaugugcuucuuucuu

SEQ ID NO: 28 - RNA, 21 nt, Homo sapiens
uucuuagagcaaacuacucaa

SEQ ID NO: 29 - RNA, 21 nt, Homo sapiens
uuuucuuagagcaaacuacuc

SEQ ID NO: 30 - RNA, 21 nt, Homo sapiens
auucuugucuuuuucuuagag

SEQ ID NO: 31 - RNA, 21 nt, Homo sapiens
uucacauugcccuuugaaguu

SEQ ID NO: 32 - RNA, 21 nt, Homo sapiens
aaaucaucacaguagauacua

SEQ ID NO: 33 - RNA, 21 nt, Homo sapiens
aauaucauuuaaaaucaucac

SEQ ID NO: 34 - RNA, 21 nt, Homo sapiens
aaaaauaucauuuaaaaucau

SEQ ID NO: 35 - RNA, 21 nt, Homo sapiens
ucagaaaggauucauacagcu

SEQ ID NO: 36 - RNA, 21 nt, Homo sapiens
uuucagaaaggauucauacag

SEQ ID NO: 37 - RNA, 21 nt, Homo sapiens
aguguaagaugaaguuuccac

SEQ ID NO: 38 - RNA, 21 nt, Homo sapiens
aaguguaagaugaaguuucca

SEQ ID NO: 39 - RNA, 21 nt, Homo sapiens
uuacagaugucuguagaagug

SEQ ID NO: 40 - RNA, 21 nt, Homo sapiens
uuauucucuguuacagauguc

SEQ ID NO: 41 - RNA, 21 nt, Homo sapiens
aagcuuuauucucuguuacag

SEQ ID NO: 42 - RNA, 21 nt, Homo sapiens
uauaggauggagaaagaacag

SEQ ID NO: 43 - RNA, 21 nt, Homo sapiens
agagauaauugaagagaagug

SEQ ID NO: 44 - RNA, 21 nt, Homo sapiens
auucuaguccaagaaucugca

SEQ ID NO: 45 - RNA, 21 nt, Homo sapiens
acguuuaggcauucuagucca

SEQ ID NO: 46 - RNA, 21 nt, Homo sapiens
uguuguacgaauuacagagca

SEQ ID NO: 47 - RNA, 21 nt, Homo sapiens
uagcaaaagcagaauacagua

SEQ ID NO: 48 - RNA, 21 nt, Homo sapiens
aauagcaaaagcagaauacag

SEQ ID NO: 49 - RNA, 21 nt, Homo sapiens
acauuugugcuuucucuucgc

SEQ ID NO: 50 - RNA, 21 nt, Homo sapiens
aauguaugcauuaucgaugcg

SEQ ID NO: 51 - RNA, 21 nt, Homo sapiens
ucaaauguaugcauuaucgau

SEQ ID NO: 52 - RNA, 21 nt, Homo sapiens
uucaaauguaugcauuaucga

SEQ ID NO: 53 - RNA, 21 nt, Homo sapiens
uuauggaaaaguucaaaugua

SEQ ID NO: 54 - RNA, 21 nt, Homo sapiens
uuuuauuauggaaaaguucaa

SEQ ID NO: 55 - RNA, 21 nt, Homo sapiens
aaauuuuauuauggaaaaguu

SEQ ID NO: 56 - RNA, 21 nt, Homo sapiens
agaaaaaauuuuauuauggaa

SEQ ID NO: 57 - RNA, 21 nt, Homo sapiens
uagaaaaaauuuuauuaugga

SEQ ID NO: 58 - RNA, 21 nt, Homo sapiens
uagauacacaaauuccuacug

SEQ ID NO: 59 - RNA, 21 nt, Homo sapiens
aaguagauacacaaauuccua

SEQ ID NO: 60 - RNA, 21 nt, Homo sapiens
aaaguagauacacaaauuccu

SEQ ID NO: 61 - RNA, 21 nt, Homo sapiens
ucuuuuuccuuuuuguauccu

SEQ ID NO: 62 - RNA, 21 nt, Homo sapiens
aaccuuaagggaagaaaccac

SEQ ID NO: 63 - RNA, 21 nt, Homo sapiens
aaaccuuaagggaagaaacca

SEQ ID NO: 64 - RNA, 21 nt, Homo sapiens
uuuacuuaaucuuugcuucca

SEQ ID NO: 65 - RNA, 21 nt, Homo sapiens
ucacuuuacuuaaucuuugca

SEQ ID NO: 66 - RNA, 21 nt, Homo sapiens
ucacuuuacuuaaucuuugcc

SEQ ID NO: 67 - RNA, 21 nt, Homo sapiens
ucacuuuacuuaaucuuugcg

SEQ ID NO: 68 - RNA, 21 nt, Homo sapiens
ucacuuuacuuaaucuuugcu

(19) United States
(12) Patent Application Publication
(10) Pub. No.: US 2020/0352979 A1
(43) Pub. Date: Nov. 12, 2020

(54) COMPOSITIONS AND METHODS FOR TREATMENT OF OBESITY AND OBESITY-RELATED DISORDERS

(71) Applicant: John Mansell, Gillette, WY (US)

(72) Inventor: John Mansell, Gillette, WY (US)

(21) Appl. No.: 16/965,988

(22) PCT Filed: Feb. 1, 2019
(86) PCT No.: PCT/US2019/016419

§ 371 Date: Jul. 29, 2020

Related U.S. Application Data
(60) Provisional application No. 62/625,688, filed on Feb. 2, 2018.

(51) Int. Cl. A61K 31/713; A61P 3/04; C12N 15/113

(52) U.S. Cl. CPC A61K 31/713; C12N 15/1137; C12N 15/1136; A61P 3/04

(57) ABSTRACT

A method of inhibiting the expression of ghrelin in a cell, method comprising introducing to the cell an RNAi, the RNAi comprising a sense strand and an antisense strand, the antisense strand comprising any of SEQ ID NO: 1 to SEQ ID NO: 42, and the sense strand being complementary to the antisense strand. Also, a method of inhibiting the expression of ghrelin o-acyltransferase in a cell, the method comprising introducing to the cell an RNAi, the RNAi comprising a sense strand and an antisense strand, the antisense strand comprising any of SEQ ID NO: 43 to SEQ ID NO: 68, and the sense strand being complementary to the antisense strand.

### CROSS-REFERENCE TO RELATED APPLICATIONS

This application is a filing under 35 U.S.C. 371 of International Application No. PCT/US2019/016419 filed Feb. 1, 2019, which is continuation of and claims priority to U.S. Provisional Patent Application Ser. No. 62/625,688 filed Feb. 2, 2018 by John Mansell and entitled “Compositions and Methods for Treatment of Obesity and Obesity Related Disorders” which is incorporated herein by reference as if reproduced in its entirety.

### REFERENCE TO SEQUENCE LISTING

The content of the ASCII text file of the sequence listing named “4983.01201.SL_ST25.txt” which is 10.0 KB in size and was created on Feb. 1, 2019 and electronically submitted via the USPTO's “EFS-Web” patent application and document submission system herewith is incorporated herein by reference in its entirety.

### TECHNICAL FIELD

The present disclosure relates to compositions and methods for treatment of obesity and obesity-related disorders. More particularly, the present disclosure relates to compositions and methods for the inhibition of ghrelin and ghrelin-associated molecules.

### BACKGROUND

Obesity (excessive adiposity) is a major public health problem. The results of a 2003-2004 National Health and Nutrition Examination Survey (NHANES) indicated that an estimated 66% of US adults are overweight or obese, and 17% of US children are overweight. Excessive adiposity is a serious problem, and is associated with insulin resistance, dyslipidemia, low-grade inflammation, and changes in levels of growth factor and other hormones that play a role in the development of diabetes, atherosclerosis, and some types of cancer. Furthermore, evidence is accumulating that excessive adiposity is associated with accelerated aging. Studies have shown that even a modest reduction in weight has a positive impact on cardiovascular risk factors and is associated with a reduced risk for developing type 2 diabetes mellitus and diabetes-associated complications.

Lifestyle interventions aimed at reducing calories and increasing physical activity through behavioral changes are currently recommended as the first-line approach for weight management. However, these strategies alone are less successful when compared to pharmacological interventions for maintained weight loss (6 to 12 months). Unfortunately, most of the drugs approved for the treatment of obesity have been withdrawn from use due to their side effects. A potential exists to target biomolecules integral to appetite such as the stomach-derived peptide hormone ghrelin.

Ghrelin also known as lenomorelin (INN), is a 28-amino acid, serine-3 acylated peptide hormone produced by ghrelinergic cells in the gastrointestinal tract. The amino acid sequence of human ghrelin is depicted in FIG. 1 using the IUPAC-IUB one letter notation for amino acids. FIG. 1 depicts the amino acid sequence of ghrelin (100) and identifies the serine-3 (110) that is acylated in the active protein. In addition to appetite, ghrelin promotes the differentiation of adipocytes and the preference for storage of calories in adipose tissue. At the peripheral level, ghrelin regulates glucose and lipid metabolism. In fact, ghrelin has a diabetogenic action and suppresses glucose-stimulated insulin secretion and deteriorates glucose tolerance. In addition to ghrelin's role as a key regulator of nutrient sensing, meal initiation, and appetite, ghrelin signaling also plays crucial roles in glucose and energy-homeostasis, cardioprotection, muscle atrophy, bone metabolism and cancer.

There exists an ongoing need for compositions and methods of treating excessive adiposity and disease related to same.

### SUMMARY

In an aspect, a method of inhibiting the expression of ghrelin in a cell. The method may comprise introducing to the cell an RNAi. The RNAi may comprise a sense strand and an antisense strand. The antisense strand may comprise any of SEQ ID NO: 1 to SEQ ID NO: 42. The sense strand may be complementary to the antisense strand.

In an aspect, a method of inhibiting the expression of ghrelin o-acyltransferase in a cell. The method may comprise introducing to the cell an RNAi. The RNAi may comprise a sense strand and an antisense strand. The antisense strand may comprise any of SEQ ID NO: 43 to SEQ ID NO: 68. The sense strand is complementary to the antisense strand.

In an aspect, a composition for inhibiting the expression of ghrelin in a cell. The composition may comprise an RNAi. The RNAi may comprise a sense strand and an antisense strand. The antisense strand may comprise any of SEQ ID NO: 1 to SEQ ID NO: 68. The sense strand may be complementary to the antisense strand.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 is a depiction of the amino acid sequence of human ghrelin.

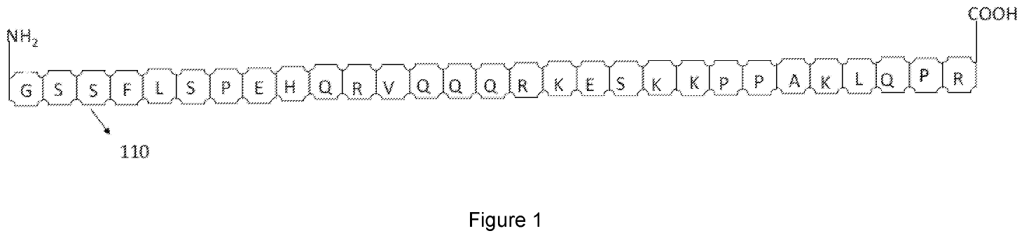

### DETAILED DESCRIPTION

Disclosed herein are methods of treating a subject suffering from obesity and obesity-related diseases, such as diabetes, by the administration of compositions and methods to inhibit, reduce or ameliorate the effect of ghrelin expression.

The term “subject” as used herein, refers to an animal which is the object of treatment, observation, or experiment. By way of example only, a subject may be, but is not limited to, a mammal including, but not limited to, a human. In some instances, the subject is a patient who is undergoing treatment for one or more medical conditions. The terms “treat,” “treating,” or “treatment,” as used herein, include alleviating, abating, or ameliorating a disease or condition, or symptoms thereof; managing a disease or condition, or symptoms thereof; preventing additional symptoms; ameliorating or preventing the underlying metabolic causes of symptoms; inhibiting the disease or condition, e.g., arresting the development of the disease or condition; relieving the disease or condition; causing regression of the disease or condition; relieving a symptom caused by the disease or condition; and/or stopping the symptoms of the disease or condition. Treatment as used herein also encompasses any pharmaceutical or medicinal use of the compositions herein.

In an aspect, the subject is administered the compositions disclosed herein in a therapeutically effective amount sufficient for treating, preventing, and/or ameliorating one or more symptoms of a medical condition, disorder, disease, or dysfunction, for example obesity or an obesity-related disorder. Hereinafter, for simplicity, the unwanted condition which has been used interchangeably with the terms medical condition, disorder, disease, and dysfunction are collectively referred to as the “medical condition.” As used herein, amelioration of the symptoms of the medical condition by administration of a particular composition of the type disclosed herein refers to any lessening, whether lasting or transient, which can be attributed to or associated with administration of compositions of the type disclosed herein.

As used herein, a “therapeutically effective amount” means a sufficient amount of the compositions disclosed herein to treat, prevent, and/or ameliorate one or more symptoms of the medical condition. It also may include a safe and tolerable amount of the compositions disclosed herein, as based on industry and/or regulatory standards. As will be understood by the ordinarily skilled artisan, an amount that proves to be a “therapeutically effective amount” in a given instance, for a particular subject, may not be effective for 100% of subjects similarly treated for the medical condition under consideration, even though such dosage is deemed a “therapeutically effective amount” by ordinarily skilled practitioners. The therapeutically effective amount for a particular individual may vary depending on numerous factors such as the nature of the medical condition, severity of the medical condition, subject weight, subject age, and the general health of the subject. It is contemplated that the therapeutically effective amount may be optimized by one or more healthcare professionals in consideration of the particular factors affecting a subject.

In an aspect, gene silencing of ghrelin using any of the methodologies or compositions disclosed herein results in a reduction in the circulating concentration of ghrelin. As such the materials disclosed herein are collectively designated disruptors of ghrelin (DOG). The phrase “gene silencing” refers to a process by which the expression of a specific gene product is lessened or attenuated. Gene silencing can take place by a variety of pathways. Unless specified otherwise, as used herein, gene silencing refers to decreases in gene product expression that results from RNA interference (RNAi), a defined, though partially characterized pathway whereby a silencing form of RNA act in concert with host proteins (e.g., the RNA induced silencing complex, RISC) to degrade messenger RNA (mRNA) in a sequence-dependent fashion. The level of gene silencing can be measured by a variety of means, including, but not limited to, measurement of transcript levels by Northern Blot Analysis, B-DNA techniques, transcription-sensitive reporter constructs, expression profiling (e.g., DNA chips), and related technologies. Alternatively, the level of silencing can be measured by assessing the level of the protein encoded by a specific gene. This can be accomplished by performing a number of studies including Western Analysis, measuring the levels of expression of a reporter protein that has e.g., fluorescent properties (e.g., GFP) or enzymatic activity (e.g., alkaline phosphatases), or several other procedures.

In an aspect, the silencing mechanisms are induced by small interfering RNAs (siRNAs), alternatively short hairpin RNAs (shRNAs), alternatively microRNAs, or alternatively bifunctional shRNAs. The phrase “gene silencing” refers to a process by which the expression of a specific gene product is lessened or attenuated. Gene silencing can take place by a variety of pathways. In an aspect, the present disclosure contemplates the utilization of RNAi in the form of siRNA which refers to short interfering RNA or silencing RNA, is a class of double-stranded RNA molecules, that may be 19 base pairs in length, alternatively 20 base pairs in length, alternatively 21 base pairs in length, alternatively 22 base pairs in length, alternatively 23 base pairs in length, alternatively 24 base pairs in length or alternatively 25 base pairs in length. It is to be understood that the sequences of the RNAi disclosed herein may be utilized in any suitable form (e.g., shRNA, siRNA, miRNA, etc.) consistent to achieve some user and/or process goal. In an aspect, an RNAi of the type disclosed herein is in the form of shRNA. In yet another aspect, an RNAi of the type disclosed herein is in the form of siRNA.

The level of gene silencing can be measured by a variety of means, including, but not limited to, measurement of transcript levels by Northern Blot Analysis, B-DNA techniques, transcription-sensitive reporter constructs, expression profiling (e.g., DNA chips), and related technologies. Alternatively, the level of silencing can be measured by assessing the level of the protein encoded by a specific gene. This can be accomplished by performing a number of studies including Western Analysis, measuring the levels of expression of a reporter protein that has e.g., fluorescent properties (e.g., GFP) or enzymatic activity (e.g., alkaline phosphatases), or several other procedures.

In an aspect, the DOG comprises shRNA, shRNA derivatives or constructs thereof. It is contemplated that the DOGs disclosed herein utilize cellular endogenous processing machinery and allow for high potency and sustainable effects using low copy numbers resulting in less off-target effects. In an aspect, the DOGs disclosed herein result in substantial silencing of the GHRL gene and consequently the reduced production of ghrelin. Alternatively, the DOGs disclosed herein result in substantial silencing of the ghrelin o-acyltransferase (GOAT) gene required for activation of ghrelin. As used herein the term “substantial silencing” means that the mRNA of the targeted allele (e.g., of GHRL) is inhibited and/or degraded by the presence of the introduced DOG, such that expression of the targeted allele is reduced by about 10% to 100% as compared to the level of expression seen when the DOG is not present. Generally, when an allele is substantially silenced, it will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, 98%, 99% or even 100% reduction in expression as compared to the level of expression obtained when the DOG is not present.

The DOGs disclosed herein comprise nucleic acids or nucleotides. The term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides. As used herein, the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, in some aspects, the entire sugar moiety can be replaced with sterically and electronically similar structures, such as aza-sugars and carbocyclic sugar analogs. Examples of modifications in a base moiety that may be utilized in the present disclosure include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

In an aspect, the DOG is a shRNA or alternatively an shRNA construct comprising an shRNA as a component of a vector. Vectors typically comprise the DNA of a transmissible agent, into which foreign DNA (e.g., a DOG) is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid,” which generally is a self-contained molecule of double-stranded DNA, usually of bacterial origin; that can readily accept additional (foreign) DNA and which can be readily introduced into a suitable host cell.

In an aspect of the present disclosure, DOGs of the type disclosed herein may be components of and transfected as plasmid vectors encoding shRNAs transcribed by RNA pol III or modified pol II promoters but can also be delivered into mammalian cells through infection of the cell with virally produced vectors. DOGs of the type disclosed herein are capable of DNA integration and consist of two complementary RNA sequences ranging in size from about 19 base pairs (bp) to about 22 bp, alternatively 19 bp, alternatively 20 bp, alternatively 21 bp or alternatively 22 bp linked by a short loop ranging in size from about 4 bp to about 11 bp, alternatively 4 bp, alternatively 5 bp, alternatively 6 bp, alternatively 7 bp, alternatively 8 bp or alternatively 9 bp. shRNA loops suitable for use in the disclosed sequences may be designed using ab initio or experimental design parameters or may be obtained from libraries of hairpin structures such as from retroviral hairpin-loop-libraries in an RNAi reporter assay. In an aspect, the shRNA loop sequence comprises the nt sequence 5′-UGUGCUU-3′. Without wishing to be limited by theory, a DOG of the type disclosed herein binds to the target mRNA and is incorporated into the RISC complex for target-specific mRNA degradation. In such aspects, translation of the targeted gene (e.g., GHRL) is reduced to the amounts disclosed herein. In yet another aspect, the targeted gene (e.g., GHRL) has reduced translation and a reduced amount of gene product is formed (e.g., ghrelin).

In an alternative aspect, DOGs of the type disclosed herein may be introduced into a cell (e.g., human cell) by any method that is now known or that comes to be known and that from reading this disclosure, persons skilled in the art would determine would be useful in connection with the present disclosure in enabling RNAi to cross the cellular membrane. These methods include, but are not limited to, any manner of transfection, such as, for example, transfection employing DEAE-Dextran, calcium phosphate, cationic lipids, liposomes, micelles, manipulation of pressure, microinjection, electroporation, immunoporation, use of vectors such as viruses, plasmids, cosmids, bacteriophages, cell fusions, and coupling of the polynucleotides to specific conjugates or ligands such as antibodies, antigens, or receptors, passive introduction, adding moieties to the siRNA that facilitate its uptake, and the like. In an aspect, a DOG of the type disclosed herein is introduced to a cell (e.g., human cell) by passive uptake. As presented in the Sequence Listing, Sequence ID No. 01 through Sequence ID No. 42 are representative of the sense strand of the DOGs of the type disclosed herein that inhibit the expression of ghrelin or alternatively inhibits the activation of ghrelin Consequently, the present disclosure contemplates the use of shRNA comprising the sense strands of any of Sequence ID No. 01 through Sequence ID No. 42 and its complementary strand. In an aspect, the shRNAs of the present disclosure comprise the polynucleotides of any of Sequence ID No 01 through Sequence ID No. 42 and its perfect complement, alternatively the polynucleotides of any of Sequence ID No 01 through Sequence ID No. 42 and a complementary strand. As presented in the Sequence Listing, Sequence ID No. 43 through Sequence ID No. 68 are representative of the sense strand of the DOGs of the type disclosed herein that inhibit the expression of GOAT. Consequently, the present disclosure contemplates the use of shRNA comprising the sense strands of any of Sequence ID No. 43 through Sequence ID No. 68 and its complementary strand. In an aspect, the shRNAs of the present disclosure comprise the polynucleotides of any of Sequence ID No 43 through Sequence ID No. 68 and its perfect complement, alternatively the polynucleotides of any of Sequence ID No 43 through Sequence ID No. 68 and a complementary strand. The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated.

Perfect complementarity or 100% complementarity refers to the situation in which each nucleotide unit of one polynucleotide strand can hydrogen bond with a nucleotide unit of a second polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity

In an aspect, a DOG comprises a shRNA, a functional variant thereof; or combinations thereof. In some aspects, a functional variant of an shRNA disclosed herein comprises at least 70% sequence identity with any sequence disclosed herein, or alternatively at least 75% sequence identity with any sequence disclosed herein, alternatively at least 80% sequence identity with any sequence disclosed herein, alternatively at least 85% sequence identity with any sequence disclosed herein, alternatively at least 90% sequence identity with any sequence disclosed herein or alternatively at least 95% sequence identity with any sequence disclosed herein.

In general, “identity” refers to an exact nucleotide-to-nucleotide correspondence of two oligonucleotides or polynucleotides sequences. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as Wisconsin Sequence Analysis Package, Version 8 (available from Genetics Computer Group, Madison, Wis.) for example, the BESTFIT, FASTA and GAP programs, which rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Appropriate hybridization conditions may be defined using any suitable methodology.

In some aspects, one or more of the nucleotides present in the shRNA may be modified to achieve one or more user and/or process goals, such as increased stability. Modified bases refer to nucleotide bases such as, for example, adenine, guanine, cytosine, thymine, uracil, xanthine, inosine, and queuosine that have been modified by the replacement or addition of one or more atoms or groups. Some examples of types of modifications that can comprise nucleotides that are modified with respect to the base moieties suitable for use in the present disclosure include but are not limited to, alkylated, halogenated, thiolated, aminated, amidated, or acetylated bases, individually or in combination. More specific examples of modified bases suitable for use in the present disclosure include without limitation and for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyluridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Herein modified nucleotides also refer to those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles. The term nucleotide is also meant to include what are known in the art as universal bases. By way of example, universal bases include but are not limited to 3-nitropyrrole, 5-nitromdole, or nebularine. The term “nucleotide” is also meant to include the N3′ to P5′ phosphoramidate, resulting from the substitution of a ribosyl 3′ oxygen with an amine group. Further, the term nucleotide also includes those species that have a detectable label, such as for example a radioactive or fluorescent moiety, or mass label attached to the nucleotide.

Without wishing to be limited by theory, utilization of a DOG of the type disclosed herein would diminish efficient transcription of ghrelin mRNA, reduce successful movement of guide strand mRNA to translation and interfere with efficient translation of mRNA which produces ghrelin. Without wishing to be limited by theory, utilization of a DOG of the type disclosed herein would diminish efficient transcription of ghrelin o-acyltransferase mRNA, reduce successful movement of guide strand mRNA to translation and interfere with efficient translation of mRNA which produces ghrelin o-acyltransferase.

In an aspect, a DOG of the type disclosed herein may be a component of a kit and may be formulated as a pharmaceutical composition. For example, the DOG may be associated with colloidal drug carrier systems such as micellar solutions, vesicle and liquid crystal dispersions, as well as nanoparticle dispersions consisting of small particles ranging in particle size from about 10 nm to about 400 nm diameter. In some aspects, a kit comprising a DOG of the type disclosed herein may further comprise a buffer, diluent, penetration enhancer, carrier compound, and/or pharmaceutically acceptable carrier or excipient.

In an aspect, a DOG of the type disclosed herein is associated with a liposome or niosome. Liposomes are a form of vesicles that consist either of many, few or just one phospholipid bilayers. The polar character of the liposomal core enables polar drug molecules to be encapsulated. Amphiphilic and lipophilic molecules are solubilized within the phospholipid bilayer according to their affinity towards the phospholipids. Participation of nonionic surfactants instead of phospholipids in the bilayer formation results in niosomes. shRNA of the type disclosed herein (i.e., DOGs) can be incorporated without loss of their activity within the hydrophobic domain of vesicle membranes, acting as a size-selective filter, only allowing passive diffusion of small solutes such as ions, nutrients and antibiotics. Thus, DOGs may be encapsulated in a nanocage and are effectively protected from premature degradation by proteolytic enzymes. In an aspect, a material suitable for use in the present disclosure is an encapsulated DOG.

In an aspect the DOG is associated with a dendrimer. Dendrimers are nanometer-sized, highly branched and monodisperse macromolecules with symmetrical architecture. They consist of a central core, branching units and terminal functional groups. The core together with the internal units, determine the environment of the nanocavities and consequently their solubilizing properties, whereas the external groups the solubility and chemical behavior of these polymers. Targeting effectiveness is affected by attaching targeting ligands at the external surface of dendrimers, while their stability and protection from the Mononuclear Phagocyte System (MPS) is being achieved by functionalization of the dendrimers with polyethylene glycol chains (PEG). In an aspect, a material suitable for use in the present disclosure is a DOG associated with a dendrimer, alternatively a DOG associated with a dendrimer comprising targeting ligands.

In an aspect, the DOG is associated with a liquid crystal. Liquid crystals combine the properties of both liquid and solid states. They can be made to form different geometries, with alternative polar and non-polar layers (i.e., a lamellar phase) where aqueous drug solutions can be included. In an aspect, a material suitable for use in the present disclosure is a DOG associated with a liquid crystal.

In an aspect, the DOG is associated with a nanoparticle. Nanoparticles (including nanospheres and nanocapsules of size 10-200 nm) are in the solid state and are either amorphous or crystalline. They are able to adsorb and/or encapsulate a drug, thus protecting it against chemical and enzymatic degradation. Nanocapsules are vesicular systems in which the drug is confined to a cavity surrounded by a unique polymer membrane, while nanospheres are matrix systems in which the drug is physically and uniformly dispersed. Nanoparticles as drug carriers can be formed from both biodegradable polymers and non-biodegradable polymers. In recent years, biodegradable polymeric nanoparticles have attracted considerable attention as potential drug delivery devices in view of their applications in the controlled release of drugs, in targeting particular organs/tissues, as carriers of DNA in gene therapy, and in their ability to deliver proteins, peptides and genes through the peroral route. In an aspect, a material suitable for use in the present disclosure is a DOG associated with a nanoparticle.

In an aspect, the DOG is associated with a hydrogel. Hydrogels are three-dimensional, hydrophilic, polymeric networks capable of imbibing large amounts of water or biological fluids. The networks are composed of homopolymers or copolymers and are insoluble due to the presence of chemical crosslinks (tie-points, junctions), or physical crosslinks, such as entanglements or crystallites. Hydrogels exhibit a thermodynamic compatibility with water, which allows them to swell in aqueous media. They are used to regulate drug release in reservoir-based, controlled release systems or as carriers in swellable and swelling-controlled release devices. On the forefront of controlled drug delivery, hydrogels as enviro-intelligent and stimuli-sensitive gel systems modulate release in response to pH, temperature, ionic strength, electric field, or specific analyte concentration differences. In these systems, release can be designed to occur within specific areas of the body (e.g., within a certain pH of the digestive tract) or also via specific sites (adhesive or cell-receptor specific gels via tethered chains from the hydrogel surface). Hydrogels as drug delivery systems can be very promising materials if combined with the technique of molecular imprinting. In an aspect, a material suitable for use in the present disclosure is a DOG associated with a hydrogel.

DOGS associated with one or more of the delivery systems disclosed herein may be considered as packaged therapeutic agents and are herein denoted “p-DOGs.” p-DOGs may be further modified to improve properties such as bioavailiability by modification of the packaging (e.g.,) using any suitable methodology (e.g., conjugation with a targeting molecule).

In an aspect, a DOG of the type disclosed herein may be administered to a subject suffering from obesity or an obesity-related disorder. The choice of a delivery route for a DOG of the type disclosed herein is driven by patient acceptability, the properties of the DOG (such as its solubility), access to a disease location, or effectiveness in dealing with the specific disease. In an aspect, the drug delivery route is the peroral route. In an aspect, a DOG of the type disclosed herein is delivered via intravenous, inhalation, intrathecal or subcutaneous injection for e.g., following mixing with saline.

In an aspect, a DOG of the type disclosed herein is administered via pulmonary delivery. Pulmonary delivery may be affected in a variety of ways—via aerosols, metered dose inhaler systems (MDIs), powders (dry powder inhalers, DPIs) and solutions (nebulizers), all of which may contain nanostructures such as liposomes, micelles, nanoparticles and dendrimers. Pulmonary drug delivery offers both local targeting for the treatment of respiratory diseases and increasingly appears to be a viable option for the delivery of drugs systemically.

In an aspect, a DOG of the type disclosed herein is delivered transdermally. Transdermal drug delivery avoids problems such as gastrointestinal irritation, metabolism, variations in delivery rates and interference due to the presence of food. It is also suitable for unconscious patients. The technique is generally non-invasive and aesthetically acceptable and can be used to provide local delivery over several days. In an aspect of the present disclosure, DOGs of the type disclosed herein may be self-administered, for example as is conventionally performed when diabetics self-administer insulin.

In an aspect, a DOG of the type disclosed herein is delivered parenterally such as through the use of trans-tissue and local delivery systems. The aim of such systems is to produce an elevated pharmacological effect, while minimizing systemic, administration-associated toxicity. Trans-tissue systems include: drug-loaded gelatinous gels, which are formed in-situ and adhere to resected tissues, releasing drugs, proteins or gene-encoding adenoviruses; antibody-fixed gelatinous gels (cytokine barrier) that form a barrier, which, on a target tissue could prevent the permeation of cytokines into that tissue; cell-based delivery, which involves a gene-transduced oral mucosal epithelial cell (OMEC)-implanted sheet; device-directed delivery—a rechargeable drug infusion device that can be attached to the targeted site, such as by intrathecal or intraperitoneal injection.

In an aspect, a method of the present disclosure comprises administering to a subject in need thereof a DOG of the type disclosed herein. In another aspect, a method of the present disclosure comprises introducing at least one DOG of the type disclosed herein to a cell such as a human cell. Alternatively a method of the present disclosure comprises introducing a plurality of DOGs of the type disclosed herein to a cell such as a human cell. Alternatively, a method of the present disclosure comprises introducing a single DOG of the type disclosed herein to a cell, such as a human cell.

Having described various compositions and methods herein, exemplary embodiments or aspects can include, but are not limited to:

A first aspect is a method of inhibiting the expression of ghrelin in a cell, the method comprising introducing to the cell an RNAi, wherein the RNAi comprises a sense strand and an antisense strand, wherein the antisense strand comprises any of SEQ ID NO: 1 to SEQ ID NO: 42, and wherein the sense strand is complementary to the antisense strand.

A second aspect is the method of the first aspect, wherein the antisense strand and the sense strand of the RNAi are each 19 to 25 nucleotides in length.

A third aspect is the method of one of the first through the second aspects, wherein the RNAi comprises at least one modified nucleotide.

A fourth aspect is the method of one of the first through the third aspects, wherein the RNAi is associated with a dendrimer.

A fifth aspect is the method of one of the first through the fourth aspects, wherein the RNAi is associated with a nanoparticle.

A sixth aspect is the method of one of the first through the fifth aspects, wherein the RNAi is siRNA, microRNA, shRNA, or a combination thereof.

A seventh aspect is the method of one of the first through the sixth aspects, wherein the RNAi is encapsulated.

An eighth aspect is the method of one of the first through the seventh aspects, wherein the cell is a human cell.

A ninth aspect is the method of one of the first through the eighth aspects, wherein said introducing is via transfection.

A tenth aspect is the method of one of the first through the ninth aspects, wherein said introducing is via passive uptake.

An eleventh aspect is a method of inhibiting the expression of ghrelin o-acyltransferase in a cell, the method comprising introducing to the cell an RNAi, wherein the RNAi comprises a sense strand and an antisense strand, wherein the antisense strand comprises any of SEQ ID NO: 43 to SEQ ID NO: 68, and wherein the sense strand is complementary to the antisense strand.

A twelfth aspect is the method of the eleventh aspect, wherein the antisense strand and the sense strand of the RNAi are each 19 to 25 nucleotides in length.

A thirteenth aspect is the method of one of the eleventh through the twelfth aspects, wherein the RNAi comprises at least one modified nucleotide.

A fourteenth aspect is the method of one of the eleventh through the thirteenth aspects, wherein the RNAi is associated with a dendrimer.

A fifteenth aspect is the method of one of the eleventh through the fourteenth aspects, wherein the RNAi is associated with a nanoparticle.

A sixteenth aspect is the method of one of the eleventh through the fifteenth aspects, wherein the RNAi is siRNA, microRNA, shRNA, or a combination thereof.

A seventeenth aspect is the method of one of the eleventh through the sixteenth aspects, wherein the RNAi is encapsulated.

An eighteenth aspect is the method of one of the eleventh through the seventeenth aspects, wherein the cell is a human cell.

A nineteenth aspect is the method of one of the eleventh through the eighteenth aspects, wherein said introducing is via transfection.

A twentieth aspect, the method of one of the eleventh through the nineteenth aspects, wherein said introducing is via passive uptake.

A twenty-first aspect is a composition for inhibiting the expression of ghrelin in a cell, the composition comprising an RNAi, wherein the RNAi comprises a sense strand and an antisense strand, wherein the antisense strand comprises any of SEQ ID NO: 1 to SEQ ID NO: 68, and wherein the sense strand is complementary to the antisense strand.

A twenty-second aspect is the composition of the twenty-first aspect, further comprising a buffer, diluent, penetration enhancer, carrier compound, and/or pharmaceutically acceptable carrier or excipient.

What is claimed is:

1. A method of inhibiting the expression of ghrelin in a cell, the method comprising introducing to the cell an RNAi, wherein the RNAi comprises a sense strand and an antisense strand, wherein the antisense strand comprises any of SEQ ID NO: 1 to SEQ ID NO: 42, and wherein the sense strand is complementary to the antisense strand.

2. The method of claim 1, wherein the antisense strand and the sense strand of the RNAi are each 19 to 25 nucleotides in length.

3. The method of claim 1, wherein the RNAi comprises at least one modified nucleotide.

4. The method of claim 1, wherein the RNAi is associated with a dendrimer.

5. The method of claim 1, wherein the RNAi is associated with a nanoparticle.

6. The method of claim 1, wherein the RNAi is siRNA, microRNA, shRNA, or a combination thereof.

7. The method of claim 1, wherein the RNAi is encapsulated.

8. The method of claim 1, wherein the cell is a human cell.

9. The method of claim 1, wherein said introducing is via transfection.

10. The method of claim 1, wherein said introducing is via passive uptake.

11. A method of inhibiting the expression of ghrelin o-acyltransferase in a cell, the method comprising introducing to the cell an RNAi, wherein the RNAi comprises a sense strand and an antisense strand, wherein the antisense strand comprises any of SEQ ID NO: 43 to SEQ ID NO: 68, and wherein the sense strand is complementary to the antisense strand.

12. The method of claim 11, wherein the antisense strand and the sense strand of the RNAi are each 19 to 25 nucleotides in length.

13. The method of claim 11, wherein the RNAi comprises at least one modified nucleotide.

14. The method of claim 11, wherein the RNAi is associated with a dendrimer.

15. The method of claim 11, wherein the RNAi is associated with a nanoparticle.

16. The method of claim 11, wherein the RNAi is siRNA, microRNA, shRNA, or a combination thereof.

17. The method of claim 11, wherein the RNAi is encapsulated.

18. The method of claim 11, wherein the cell is a human cell.

19. The method of claim 11, wherein said introducing is via transfection.

20. The method of claim 11, wherein said introducing is via passive uptake.

21. A composition for inhibiting the expression of ghrelin in a cell, the composition comprising an RNAi, wherein the RNAi comprises a sense strand and an antisense strand, wherein the antisense strand comprises any of SEQ ID NO: 1 to SEQ ID NO: 68, and wherein the sense strand is complementary to the antisense strand.

22. The composition of claim 21, further comprising a buffer, diluent, penetration enhancer, carrier compound, and/or pharmaceutically acceptable carrier or excipient.
